(19) United States
(12) Patent Application Publication
(10) Pub. No.: US 2005/0143649 A1
(43) Pub. Date: Jun. 30, 2005

(54) System for detecting travel state of capsule endoscope in subject

(75) Inventors: Tetsuo Minai, Tokyo (JP); Hatsuo Shimizu, Tokyo (JP)

Correspondence Address: Thomas Spinelli;Scully, Scott, Murphy & Presser, 400 Garden City Plaza, Garden City, NY, 11530, US

(73) Assignee: OLYMPUS CORPORATION, TOKYO (JP)

(21) Appl. No.: 11/019,028

(22) Filed: Dec. 21, 2004

(30) Foreign Application Priority Data
Dec. 26, 2003 (JP) 2003-435557

(51) Int. Cl. A61B 5/05

(52) U.S. Cl. 600/410

(57) ABSTRACT

A system includes a device that is swallowed, passes through a subject, and includes a magnetic field generator generating a constant magnetic field; and a travel state detector. The travel state detector includes a magnetic detector, disposed in a fixed relative position to the subject, detecting an intensity of a constant magnetic field output from the magnetic field generator. The travel state detector also includes a position processor calculating a position of the device in the subject based on the intensity of the magnetic field detected by the magnetic detector, and a travel state processor determining a travel state of the device in the subject based on the position calculated by the position processor.

### CROSS-REFERENCE TO RELATED APPLICATIONS

This application claims the benefit of priority of Japanese Patent Application No. 2003-435557 filed on Dec. 26, 2003, and the disclosure of which is incorporated herein by its entirety.

### BACKGROUND OF THE INVENTION

1) Field of the Invention

The present invention relates to a system for detecting a travel state of a device in a subject, and the system includes the device to be swallowed and pass naturally through inside the subject, and a travel state detector that is disposed on the outside of the subject and obtains information of the position of the device in the subject.

2) Description of the Related Art

In recent years, in the field of endoscopes, a swallowable capsule endoscope has been proposed. The capsule endoscope has an image capturing function and a radio communication function. The capsule endoscope has the function of traveling in the body cavity, for example, in the organs such as the stomach and the small intestine with peristalsis of the organs and sequentially capturing images for a period of time since the capsule endoscope is swallowed from the mouth of a subject for inspection (examination) until it is naturally excreted.

Image data captured in the body by the capsule endoscope as the capsule endoscope travels in the body cavity is sequentially transmitted by radio communication to the outside and stored into a memory provided on the outside. The subject can freely move throughout the period after he/she swallows the capsule endoscope until it is excreted by carrying a receiver having a radio communication function and a storing function. After the capsule endoscope is excreted, a doctor or nurse can display the images of the organs on a display based on the image data stored in the memory and make a check.

A capsule endoscope has been proposed in which the receiver has the function of detecting the position of the capsule endoscope in the subject to capture, for example, an endoscope image of a specific organ in the subject. As an example of a capsule endoscope system having the position detecting function, a capsule endoscope system using the radio communication function provided in the capsule endoscope is known. Specifically, the system has a configuration that a receiver provided on the outside of a subject has a plurality of antenna elements, and has the function of receiving a radio signal transmitted from the capsule endoscope by the plurality of antenna elements and, based on intensities received by the antenna elements, detecting the position of the capsule endoscope in the subject (see Japanese Patent Application Laid-open No. 2003-19111, for example).

It is also possible to determine the travel state of the capsule endoscope using a position detecting mechanism. For example, the travel speed can be determined based on a positional change in a predetermined time. The intervals of image pick-up inside the subject can be adjusted based on the travel speed of the capsule endoscope. In other words, images of inside the subject can be acquired at constant distance intervals regardless of the travel speed of the capsule endoscope, by making the image pick-up interval small in a region where the travel speed of the capsule endoscope is high and making the image pick-up interval large in a region where the travel speed is low.

### SUMMARY OF THE INVENTION

It is an object of the present invention to at least solve the problems in the conventional technology.

A system according to one aspect of the present invention includes a device that is swallowed, passes through a subject, and includes a magnetic field generator generating a constant magnetic field; and a travel state detector. The travel state detector includes a magnetic detector, disposed in a fixed relative position to the subject, detecting an intensity of a constant magnetic field output from the magnetic field generator. The travel state detector also includes a position processor calculating a position of the device in the subject based on the intensity of the magnetic field detected by the magnetic detector, and a travel state processor determining a travel state of the device in the subject based on the position calculated by the position processor.

The other objects, features, and advantages of the present invention are specifically set forth in or will become apparent from the following detailed description of the invention when read in conjunction with the accompanying drawings.

### BRIEF DESCRIPTION OF THE DRAWINGS

FIG. 1 is a schematic view of a system for detecting a travel state of a capsule endoscope in a subject according to an embodiment;

FIG. 2 is a schematic view of a capsule endoscope as a component of the system according to the embodiment;

FIG. 3 is a schematic view of a travel state processor as a component of the system according to the embodiment;

FIG. 4 is a flowchart of operations of the travel state processor to calculate the position of the capsule endoscope;

FIG. 5 is a schematic view of calculating the position of a capsule endoscope by the travel state processor;

FIG. 6 is a flowchart of operations of the travel state processor to derive an orientation direction of the capsule endoscope;

FIG. 7 is a schematic view of deriving the orientation direction of the capsule endoscope by the travel state information processor;

FIG. 8 is a flowchart of operations of determining a travel state of the capsule endoscope by a travel state information generator;

FIG. 9 is a schematic view of the test capsule as a component of the system according to a modification; and

FIG. 10 is a schematic view of the travel state processor as a component of the system according to the modification.

### DETAILED DESCRIPTION

Exemplary embodiments of a system for detecting a travel state of a capsule endoscope in a subject relating to the present invention will be explained in detail below with reference to the accompanying drawings. It should be noted that the drawings are schematic ones and the relation between thickness and width of each part, the thickness ratio of the parts, and the like are different from real ones. Obviously, the drawings include parts having different relations of dimensions and ratios.

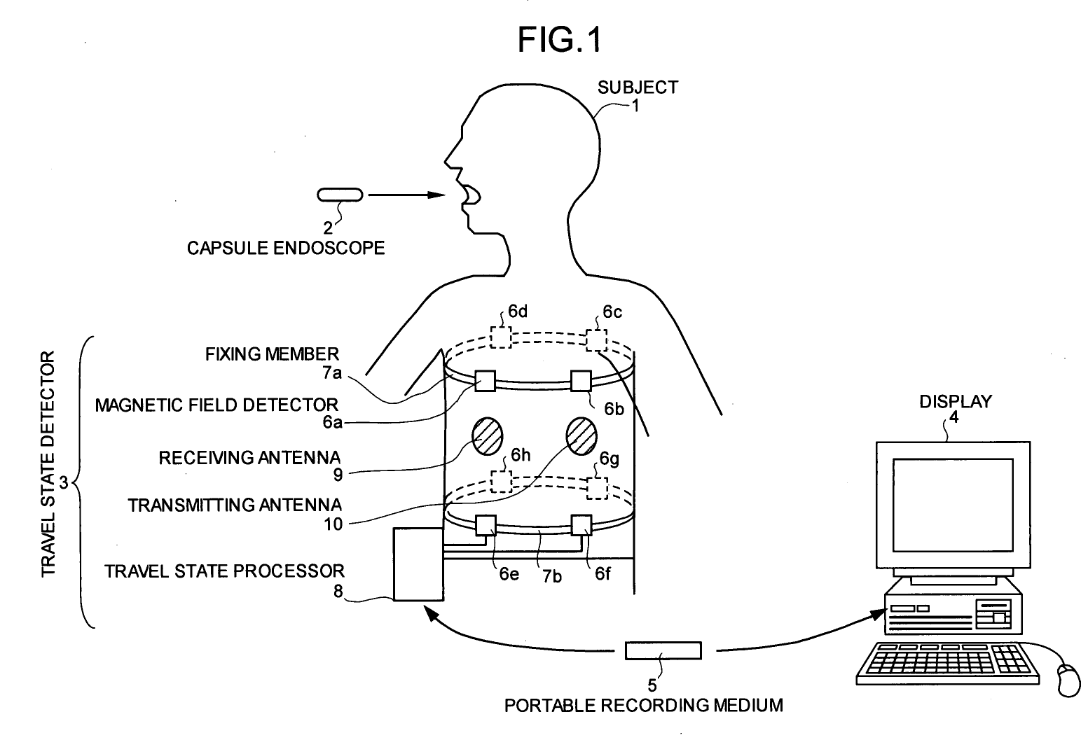

FIG. 1 is a schematic view of a system for detecting a travel state of a capsule endoscope in a subject according to an embodiment. As shown in FIG. 1, the system for detecting a travel state of a capsule endoscope according to the embodiment of the present invention includes a capsule endoscope 2 that is swallowed and passes though a subject 1 and functions as an example of a device to be traveled in a subject; a travel state detector 3 that detects, for example, the travel state of the capsule endoscope 2 in the subject 1; a display 4 that displays an image or the like of the subject 1 captured by the capsule endoscope 2; and a portable recording medium 5 for passing information between the travel state detector 3 and the display 4.

The display 4 is used for displaying an image or the like of the subject 1 captured by the capsule endoscope 2 and has a configuration like a workstation or the like that displays an image based on data obtained from the portable recording medium 5. Concretely, the display 4 may be constructed to directly display an image by a cathode-ray tube (CRT) display, a liquid crystal display, or the like or to output an image to another medium like a printer or the like.

The portable recording medium 5 can be inserted/removed to/from a travel state processor 8 that will be explained later and the display 4, and has a structure that allows retrieving and recording of information when inserted to the travel state processor 8 and the display 4. Concretely, the portable recording medium 5 is inserted into the travel state processor 8 to record information on the position of the capsule endoscope 2 while the capsule endoscope 2 travels in the body cavity of the subject 1. After the capsule endoscope 2 is excreted from the subject 1, the portable recording medium 5 is removed from the travel state processor 8 and inserted into the display 4, and the recorded data is read by the display 4. By passing data between the travel state processor 8 and the display 4 by the portable recording medium 5 such as a compact flash (trademark) memory, different from the case where the travel state processor 8 and the display 4 are connected to each other by wire, even when the capsule endoscope 2 is traveling in the subject 1, the subject 1 can move freely.

The capsule endoscope 2 travels within the subject 1 in which it is introduced and has a function of serving as a function executing unit that executes a predetermined function on the inside of the subject 1, a function of serving as a receiving apparatus that receives a radio signal transmitted from the travel state detector 3, and a function of serving as a magnetic field generator that outputs a constant magnetic field used by the travel state detector to grasp the travel state of the capsule endoscope. Hereafter, the configuration of the capsule endoscope 2 will be explained for each of components corresponding to the functions.

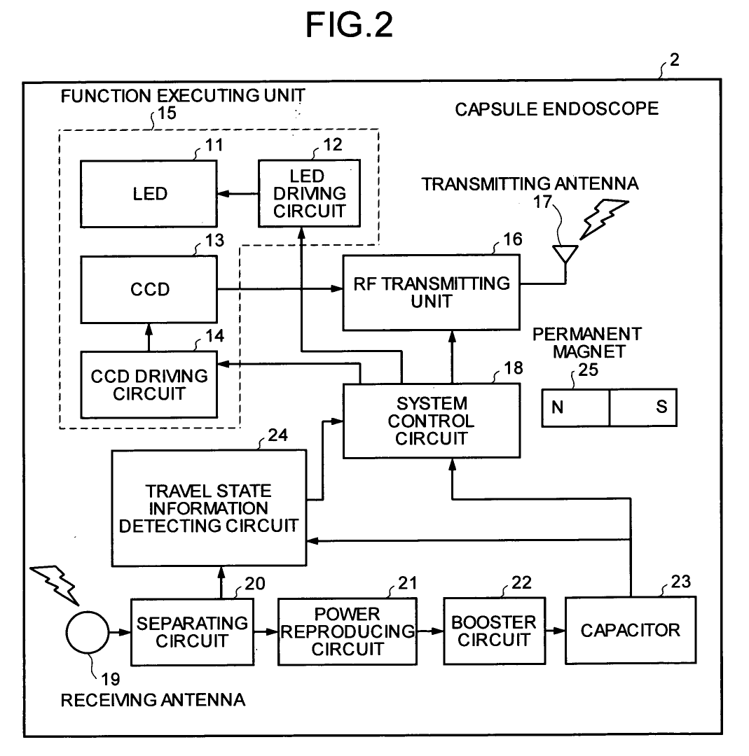

FIG. 2 is a schematic diagram of a structure of the capsule endoscope 2. First, the capsule endoscope 2 has a configuration to implement the function of serving as the function executing unit that executes the predetermined function and the function of serving as a transmitter that conducts radio transmission of information obtained by the function executing unit. Specifically, the capsule endoscope 2 includes a light-emitting diode (LED) 11 functioning as an illuminating unit that illuminates an image pickup region when capturing the image of the inside of the subject 1, an LED driving circuit 12 that controls the drive state of the LED 11, a charge-coupled device (CCD) 13 functioning as an image capturing unit that captures a reflected light image from a region illuminated by the LED 11, and a CCD driving circuit 14 that controls the drive state of the CCD 13. The LED 11, the LED driving circuit 12, the CCD 13 and the CCD driving circuit 14 are defined as a whole as a function executing unit 15 that fulfills a predetermined function.

The capsule endoscope 2 includes an RF transmitting unit 16 that modulates image data captured by the CCD 13 and generates an RF signal, a transmitting antenna unit 17 serving as a radio unit that conducts radio transmission of an RF signal output from the RF transmitting unit 16, and a system controlling circuit 18 that controls operations in the LED driving circuit 12, the CCD driving circuit 14 and the RF transmitting unit 16.

Owing to provision of these mechanisms, the capsule endoscope 2 acquires image data of an inspected region illuminated by the. LED 11 by means of the CCD 13 while the capsule endoscope is in the subject 1. The acquired image data is converted into an RF signal in the RF transmitting unit 16, and then transmitted to the outside via the transmitting antenna unit 17.

The capsule endoscope 2 has a configuration to receive a radio signal transmitted from the travel state detector 3. Specifically, the capsule endoscope 2 includes a receiving antenna unit 19 that receives a radio signal transmitted from the travel state detector 3 side, and a separating circuit 20 that separates a power supplying signal from the signal received in the receiving antenna unit 19. The capsule endoscope 2 further includes a power reproducing circuit 21 that reproduces power from the separated power supplying signal, a booster circuit 22 that boosts the reproduced power, and a capacitor 23 that stores the boosted power. The capsule endoscope 2 further includes a travel state information detecting circuit 24 that detects contents of a travel state information signal from a component separated from the power supplying signal at the separating circuit 20, and outputs the detected travel state information signal to the system control circuit 18. Here, the travel state information is information concerning the travel state of the capsule endoscope 2 and derived by the travel state processor 8. The system control circuit 18 has a function of controlling the illumination interval of the LED 11 and the image pickup interval of the CCD 13 based on the travel state information.

Owing to the provision of these mechanisms, the capsule endoscope 2 first receives a radio signal transmitted from the travel state detector 3 at the receiving antenna unit 19, and separates the power supplying signal and the travel state information signal from the received radio signal by using the separating circuit 20.

The travel state information signal separated by the separating circuit 20 is input to the system control circuit-18 via the travel state information detecting circuit 24. The system control circuit 18 controls the drive states of the LED 11, CCD 13 and the RF transmitting unit 16 based on the travel state information. Specifically, if the system control circuit 18 acquires the travel state information, for example, to the effect that the capsule endoscope 2 stops its travel in the subject 1, the system control circuit 18 exercises control to temporarily stop the drive of the CCD 13 and the LED 11 and thereby prevents pickup of duplicated image data. On the other hand, the power supplying signal is reproduced as power by the power reproducing circuit 21. The reproduced power is boosted to a potential suitable for the capacitor 23, and then stored in the capacitor 23.

The capsule endoscope 2 further has a configuration to fulfill the function of serving as the magnetic field generator. Specifically, the capsule endoscope 2 includes a permanent magnet 25 that outputs the constant magnetic field used to detect the position of the capsule endoscope 2 and detect the travel state. The permanent magnet 25 functions as a magnetic field generator in claims. The permanent magnet 25 includes a permanent magnet having such a size that the permanent magnet can be accommodated in the capsule endoscope 2, and has a function of outputting a constant magnetic field of which a fluctuation of magnetic field intensity over time is negligible. Instead of the permanent magnet 25, for example, a coil supplied with a constant current to generate a constant magnetic field may be used as the magnetic field generator. If the permanent magnet 25 is used, however, there is an advantage that driving power is unnecessary. Therefore, it is preferred to form the magnetic field generator with the permanent magnet 25.

The constant magnetic field generated by the permanent magnet 25 is represented by closed curve lines of magnetic force which start from an N pole side, travel through an external region of the capsule endoscope 2 and return to an S pole side. The intensity of the constant magnetic field represented by the lines of magnetic force can be considered to depend only on the distance from the capsule endoscope 2. In other words, the size of the permanent magnet 25 incorporated in the capsule endoscope 2 is minute enough to be negligible as compared with the distance between the capsule endoscope 2 and magnetic detectors 6a to 6h. Therefore, a magnetic field intensity P at a point that is a distance r apart from the capsule endoscope 2 is represented by the following relation by using a proportional factor α.
P=α/r³ (1)

The system according to the present embodiment detects the position of the capsule endoscope 2 based on the relation represented by Equation (1) as described later. The travel direction of the constant magnetic field output from the permanent magnet 25 has a place-dependence. As explained later, the system according to the present embodiment has a configuration also to detect a direction of a longitudinal axis of the capsule endoscope 2 (hereafter referred to as “orientation direction”) as one aspect of the position information by using the place-dependence of the travel direction of the constant magnetic field.

The travel state detector 3 will be explained. The travel state detector 3 is provided to detect the travel state of the capsule endoscope 2 in the subject 1 based on the constant magnetic field output from the capsule endoscope 2. Specifically, as shown in FIG. 1, the travel state detector 3 includes magnetic detectors 6a to 6h that detect the constant magnetic field output from the capsule endoscope 2, a fixing member 7a that fixes the magnetic detectors 6a to 6d to the subject 1, a fixing member 7b that fixes the magnetic detectors 6e to 6h to the subject 1, the travel state processor 8 that derives the position of the capsule endoscope 2 based on the magnetic field intensities detected by the magnetic detectors 6a to 6h, a receiving antenna 9 that receives a radio signal transmitted from the capsule endoscope 2, and a transmitting antenna 10 that transmits a radio signal to the capsule endoscope 2. The magnetic detectors 6a to 6h, the receiving antenna 9, and the transmitting antenna 10 are electrically connected to the travel state processor, and have a configuration to receive information from and supply information to the travel state processor 8.

The magnetic detectors 6a to 6h function to detect the magnetic field intensity and magnetic field direction at their respective locations. Specifically, each of the magnetic detectors 6a to 6h is formed by using, for example, a Magneto Impedance (MI) sensor. The MI sensor has a configuration using, for example, an FeCoSiB amorphous wire as a magneto-sensitive medium, and detects the magnetic field intensity by using the MI effect, in which the magnetic impedance of the magneto-sensitive medium is changed largely by the external magnetic field when a high frequency current is let flow through the magneto-sensitive medium. A different magnetic field sensor may be used as each of the magnetic detectors 6a to 6h. If the MI sensor is used, however, there is an advantage that the magnetic field detection can be conducted with a particularly high sensitivity. In the present embodiment, the magnetic detectors 6a to 6h are disposed in positions respectively forming vertexes of a cube.

The fixing members 7a and 7b are provided to fix the magnetic detectors 6a to 6h to the subject 1. Specifically, each of the fixing members 7a and 7b is formed in an annular form by using, for example, an elastic member, and has such a configuration that it is fixed in close contact to the trunk of the subject 1. The magnetic detectors 6a to 6d and the magnetic detectors 6e to 6h are fixed to the subject 1 in predetermined positions by the fixing members 7a and 7b, respectively. By fixing the fixing members 7a and 7b to the trunk of the subject 1 in close contact, the magnetic detectors 6a to 6h are disposed in fixed relative positions with respect to the subject 1.

The receiving antenna 9 is provided to receive a radio signal transmitted from the capsule endoscope 2. As explained later, the capsule endoscope 2 has a function of picking up an image within the subject 1 and transmitting the image to the outside in a wireless manner. The receiving antenna 9 has a function of receiving a radio signal transmitted from the capsule endoscope 2 and outputting it to the travel state processor 8. Specifically, the receiving antenna 9 includes, for example, a loop antenna and a fixing unit that fixes the loop antenna to the subject 1.

The transmitting antenna 10 is provided to transmit a signal generated by the travel state processor 8 to the capsule endoscope 2. As explained later, the travel state processor 8 in the present embodiment has a function of superposing the power supplying signal that serves as drive power for the capsule endoscope 2 on the travel state information signal that is information concerning the travel state of the capsule endoscope 2 and outputting a resultant signal to the capsule endoscope 2. The transmitting antenna 10 is provided to transmit these signals to the capsule endoscope 2 in a wireless manner. Specifically, the transmitting antenna 10 includes, for example, a loop antenna and a fixing unit that fixes the loop antenna to the subject 1.

The travel state processor 8 will be explained. The travel state processor 8 has a function of serving as a receiver that receives a radio signal transmitted from the capsule endoscope 2, a function of serving as a transmitter that transmits a predetermined signal to the capsule endoscope 2 in a wireless manner, and a function of deriving the position and the orientation direction of the capsule endoscope 2 and further deriving the travel state of the capsule endoscope 2. Hereafter, a configuration of the travel state processor 8 will be explained for every component corresponding to each function.

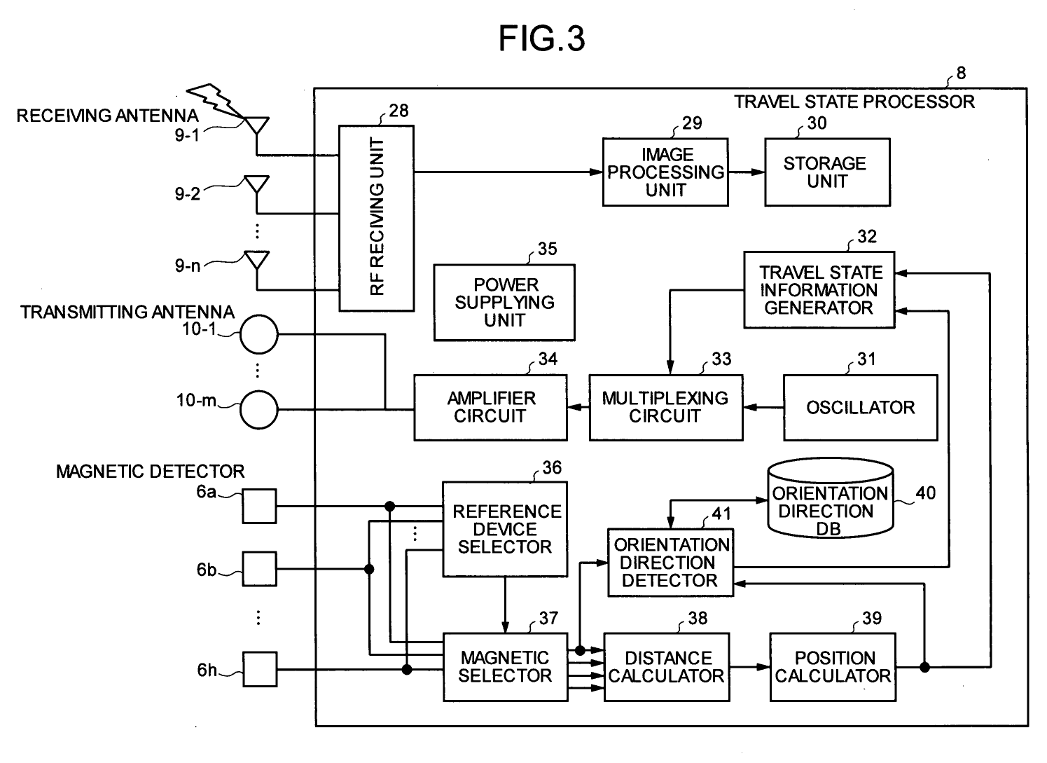

FIG. 3 is a block diagram of a general configuration of the travel state processor 8. First, the travel state processor 8 has a configuration serving as a receiving apparatus that receives image data within the subject 1 transmitted from the capsule endoscope 2 in the wireless manner. Specifically, the travel state processor 8 includes an RF receiving unit 28 that conducts predetermined processing such as demodulation on a radio signal received by a selected receiving antenna and extracts image data acquired by the capsule endoscope 2 from the radio signal, an image processing unit 29 that conducts necessary processing on output image data, and a storage unit 30 that stores image data subjected to image processing.

The travel state processor 8 has a configuration serving as a transmitter that generates the power supplying signal and the travel state information signal to be transmitted to the capsule endoscope 2 and outputs the signals to transmitting antennas 10-1 to 10-m. Specifically, as shown in FIG. 3, the travel state processor 8 includes an oscillator 31 having a function of generating the power supplying signal and a function of defining an oscillating frequency, a travel state information generator 32 that generates the travel state information signal explained later, a multiplexing circuit 33 that combines the power supplying signal and the travel state information signal, and an amplifier circuit 34 that amplifies the strength of the combined signal. The signal amplified by the amplifier circuit 34 is sent to the transmitting antennas 10-1 to 10-m, and transmitted to the capsule endoscope 2. The travel state processor 8 includes a power supplying unit 35 having a predetermined condenser or an AC power supply adaptor or the like. Components in the travel state processor 8 use power supplied from the power supplying unit 35 as driving energy.

The travel state processor 8 has a configuration serving as a position processor that calculates the position of the capsule endoscope in the subject 1 needed when generating the travel state information. Specifically, the travel state processor 8 includes a reference device selector 36 that selects a magnetic detector serving as a reference (hereafter referred to as “reference device”) from among the magnetic detectors 6a to 6h, and a magnetic selector 37 that outputs a magnetic field intensity obtained in a predetermined number of magnetic detectors based on a result of selection conducted by the reference device selector 36. The travel state processor 8 further includes a distance calculator 38 that calculates the distance between the capsule endoscope 2 and the reference device, and a position calculator 39 that calculates the position of the capsule endoscope 2 by conducting computation processing using the calculated distance and the position coordinates of the reference device used to calculate the distance.

The reference device selector 14 has the function of selecting the magnetic detector with the largest value of the detected magnetic field intensity from the magnetic detectors 6a to 6h. Concretely, the reference device selector 14 compares the magnetic field intensity values output from the magnetic detectors 6a to 6h with each other, selects the magnetic detector (reference device) that has output the largest magnetic field intensity value, and outputs information specifying the reference device (for example, information indicating which is the reference device among the magnetic detectors 6a to 6h) to the magnetic selector 37.

The magnetic selector 37 selects a plurality of magnetic detectors based on the result of selection of the reference device selector 36 and outputs the magnetic field intensities obtained by the selected magnetic detectors (selected devices) to the distance calculator 38. Concretely, the magnetic selector 37 has the function of selecting three magnetic detectors disposed in directions orthogonal to each other with respect to the reference device. Specifically, in the system according to the first embodiment, as also shown in FIG. 1, the magnetic detectors 6a to 6h are disposed so as to form vertexes of a cube, so that three magnetic detectors positioned in direction orthogonal to each other always exist for any magnetic detector, and the magnetic selector 37 has the function of selecting the three magnetic detectors as selected devices.

The distance calculator 38 calculates the distances from the reference device, and the selected devices, to the capsule endoscope 2 based on the magnetic field intensities received via the magnetic selector 37. Concretely, the distance calculator 38 has the function of calculating the distance between the magnetic detector that has detected the magnetic field intensity and the capsule endoscope 2 by performing the computing process shown by Equation (1) with respect to the input magnetic field intensity.

The position calculator 39 calculates the position of the capsule endoscope 2 by performing a predetermined computing process based on the distance between the magnetic detector selected as a reference device or the like and the test capsule 2. The position calculator 39 also has the function of calculating the position of the capsule endoscope 2 and, after that, outputting the result of calculation to the storage unit 30.

The travel state processor 8 has a configuration serving as an orientation direction detector that detects the orientation direction of the capsule endoscope 2 needed when generating the travel state information. Specifically, the travel state processor 8 includes an orientation direction database 40 that stores information concerning the orientation direction, and an orientation direction detector 41 that detects the orientation direction of the capsule endoscope 2 based on a magnetic field direction detected by a predetermined magnetic detector 6. The orientation direction database 40 stores in advance data concerning the magnetic field intensity received by the magnetic detector 6 and the orientation direction of the capsule endoscope 2 with respect to the orientational relation between the magnetic detector 6 and the capsule endoscope 2. Specific contents of operation in the orientation direction database 40 and the orientation direction detector 41 is explained in detail later.

The travel state processor 8 includes a mechanism that derives the travel state based on information concerning the calculated position of the capsule endoscope 2 and the orientation direction. Specifically, the travel state processor 8 includes the travel state information generator 32, and derives the travel state of the capsule endoscope 2 and generates the travel state information by using the travel state information generator 32.

Operation of the system according to the present embodiment is explained. Hereinafter, the operation of position calculation, orientation direction calculation, and travel state information generation concerning the capsule endoscope 2 conducted by the travel state processor 8 will be explained successively.

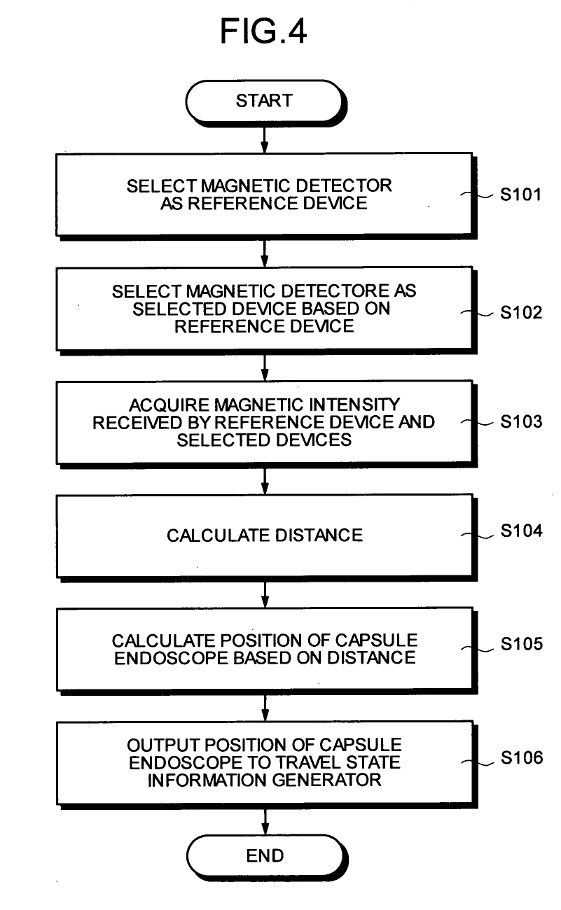

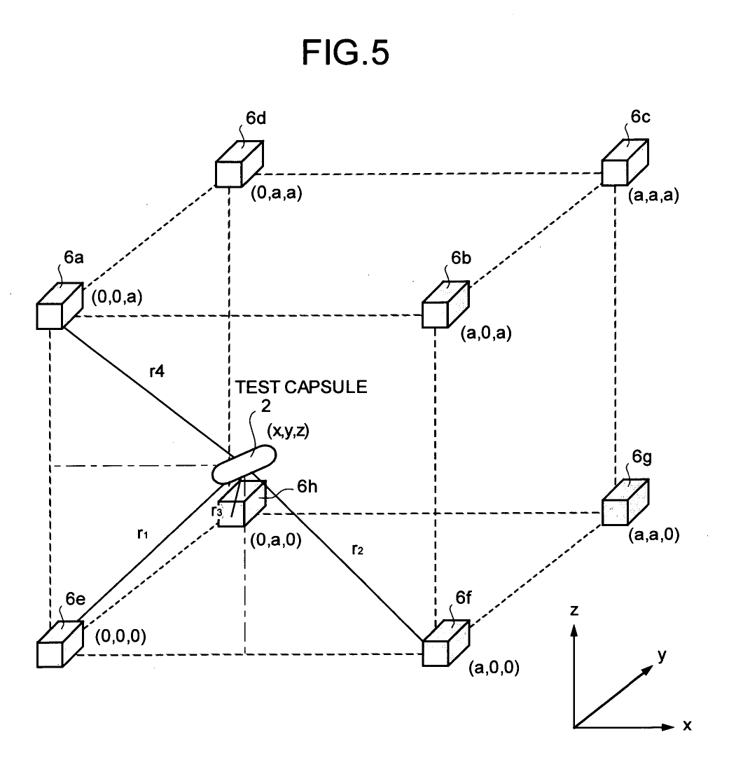

The operation of the travel state processor 8 to calculate the position of the capsule endoscope will be explained. FIG. 4 is a flowchart of the position calculation operation of the travel state processor 8, and FIG. 5 is a schematic diagram for explaining the algorithm of the position calculation. In FIG. 5, the length of one side of a cube formed by the magnetic detectors 6a to 6h is set as “a”. As is explained later, the position of the magnetic detector 6e selected as the reference device is set as the origin, the direction from the magnetic detector 6e toward the magnetic detector 6f is set as an x direction, the direction from the magnetic detector 6e toward the magnetic detector 6h is set as a y direction, and the direction from the magnetic detector 6e toward the magnetic detector 6a is set as a z direction. The positions of the magnetic detectors 6a to 6h are determined based on the xyz coordinate system, and the position of the capsule endoscope 2 in the xyz coordinate system is expressed as (x,y,z). The operation of the travel state processor 8 is explained hereinbelow by properly referring to FIGS. 4 and 5.

First, the travel state processor 8, using the reference device selector 36, selects the magnetic detector having the magnetic field intensity that is the highest among the magnetic field intensities received by the magnetic detectors 6a to 6h (step S101). In the example of FIG. 5, the magnetic detector 6e is selected as the magnetic detector sensing the highest magnetic field intensity. In the following description, it is also assumed that the magnetic detector 6e is the reference device.

The travel state processor 8 selects three devices by the magnetic selector 37 based on the reference device selected in step S101 (step S102), and outputs the magnetic field intensities obtained by the reference device and the selected devices to the distance calculator 38 (step S103). In the example of FIG. 5, the magnetic detectors 6f, 6h, and 6a are disposed in the directions orthogonal to each other with respect to the magnetic detector 6e as a reference device, so that the magnetic selector 37 selects the magnetic detectors 6f, 6h, and 6a as selected devices.

After that, the travel state processor 8 calculates the distance from the capsule endoscope 2 based on the magnetic field intensity obtained by the reference device selected in step S101 and the magnetic field intensities obtained by the devices selected in step S102 by the distance calculator 38 (step S104). Concretely, the distance calculator 38 calculates the distance by performing computation of Equation (1) using the magnetic field intensity input via the magnetic selector 37. In the example of FIG. 5, the distance calculator 38 calculates distances r₁, r₂, r₃, and r₄ between the capsule endoscope 2 and the magnetic detectors 6e, 6f, 6h, and 6a, respectively, based on the magnetic field intensities detected by the reference device and the selected devices.

The travel state processor 8 calculates the position of the capsule endoscope 2 by the computing process in the position calculator 39 (step S105). Concretely, since the position of the capsule endoscope 2 is calculated by deriving the x coordinate, y coordinate, and z coordinate of the capsule endoscope 2, the coordinates of the capsule endoscope 2 are derived by using the coordinates of the magnetic detectors 6e, 6f, 6h, and 6a and the values of distances calculated in step S104.

For example, the position coordinates (x,y,z) of the capsule endoscope 2 can be geometrically derived from the positional relations shown in FIG. 5 and, concretely, can be calculated by solving the following equations.
(x−0)²+(y−0)²+(z−0)²=r₁² (2)
(x−a)²+(y−0)²+(z−0)²=r₂² (3)
(x−0)²+(y−a)²+(z−0)²=r₃² (4)
(x−0)²+(y−0)²+(z−a)²=r₄² (5)

In Equations (2) to (5), the number of unknown letters is three so that three equations are theoretically sufficient. At the time of actual position detection, however, to suppress deterioration in precision of the position detection of the capsule endoscope 2 due to positional deviations of the magnetic detectors 6a to 6h, a distance derivation error, and the like, after solving Equations (2) to (5), the coordinates of the magnetic detector, and the like are corrected so that the values x, y, and z are determined to be unique values.

Finally, the travel state processor 8 outputs the information concerning the position of the capsule endoscope 2 calculated by the position calculator 39 to the travel state information generator 32 (step S106). The output information concerning the position of the capsule endoscope 2 is used to derive the travel state of the capsule endoscope 2 explained later.

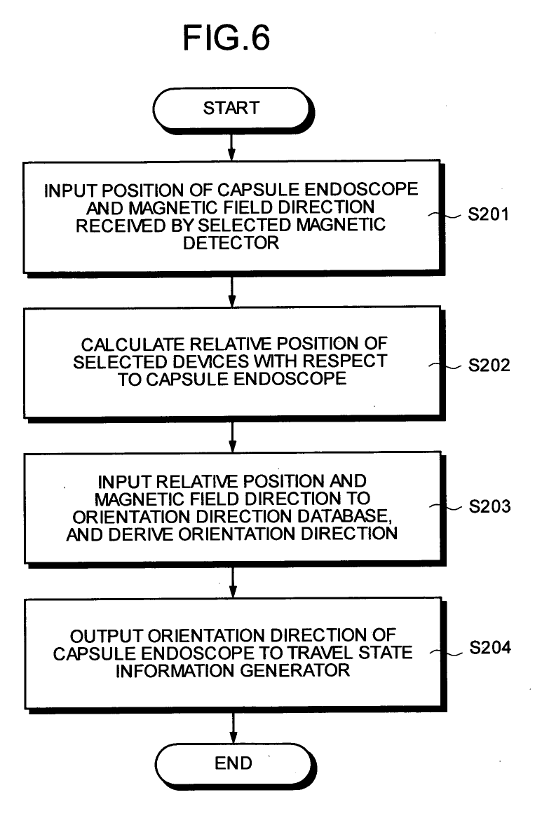

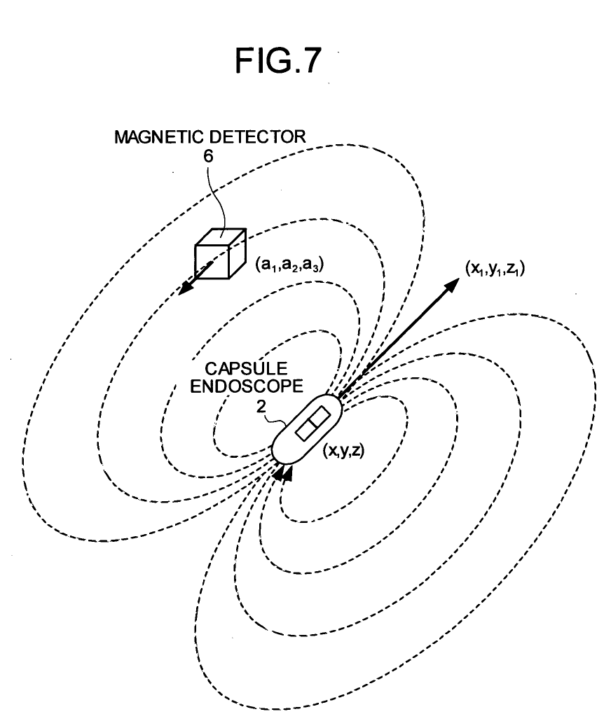

The operation of deriving the orientation direction of the capsule endoscope 2 conducted by the travel state detector 3 is explained. FIG. 6 is a flowchart of operations of deriving the orientation direction. FIG. 7 is a schematic diagram of the orientation direction deriving operation. Hereafter, the orientation direction deriving operation for the capsule endoscope 2 is explained with reference to FIGS. 6 and 7 as necessary.

First, the orientation direction detector 41 inputs the position of the capsule endoscope 2, and a magnetic field direction received by a magnetic detector 6 selected from among the magnetic detectors 6a to 6h (step S201). Any algorithm may be used to select the magnetic detector 6. In the present embodiment, however, for example, a magnetic detector 6 having the greatest received magnetic field intensity is selected. In an example shown in FIG. 7, the orientation direction detector 41 grasps coordinates (a1, a2, a3) of the selected magnetic detector 6 and a magnetic field direction represented by a direction vector indicated by an arrow.

The orientation direction detector 41 calculates a relative position of the magnetic detector 6 selected at the step S201 with respect to the capsule endoscope 2 (step S202). Specifically, the orientation direction detector 41 is supplied with the position of the capsule endoscope 2 calculated by the position calculator 39 to derive relative coordinates of the magnetic detector 6 selected at the step S201 with respect to the capsule endoscope 2. In the example shown in FIG. 7, relative position coordinates (a₁-x, a₂-y, a₃-z) of the magnetic detector with the position of the capsule endoscope 2 taken as the origin are derived based on the coordinates (a₁, a₂, a₃) of the magnetic detector 6 and the coordinates (x, y, z) of the capsule endoscope 2.

Thereafter, the orientation direction detector 41 inputs the magnetic field direction received at the step S201 and the relative position of the magnetic detector 6 selected at the step S202 to the orientation direction database 40, and acquires data concerning the orientation direction of the capsule endoscope 2 (step S203). As shown in FIG. 7, the direction of the constant magnetic field output from the permanent magnet 25 included in the capsule endoscope 2 has a property that it uniquely depends on the orientation direction of the capsule endoscope 2 and the position with respect to the capsule endoscope 2. Therefore, the orientation direction of the capsule endoscope 2, the relative coordinates with respect to the capsule endoscope 2, and the direction of the constant magnetic field at the relative coordinates are stored in the orientation direction database 40 so as to be associated with each other. Therefore, the orientation direction of the capsule endoscope 2 can be extracted by inputting the relative coordinates of the magnetic detector 6 and the detected direction of the constant magnetic field to the orientation direction database 40. In the example shown in FIG. 7, it is derived that the orientation direction of the capsule endoscope 2 is (x₁, y₁, z₁) based on the output result of the orientation direction database 40.

The orientation direction detector 41 outputs acquired information concerning the orientation direction of the capsule endoscope 2 to the travel state information generator 32 (step S204). The output information concerning the orientation direction of the capsule endoscope 2 is used to derive the next travel state of the capsule endoscope.

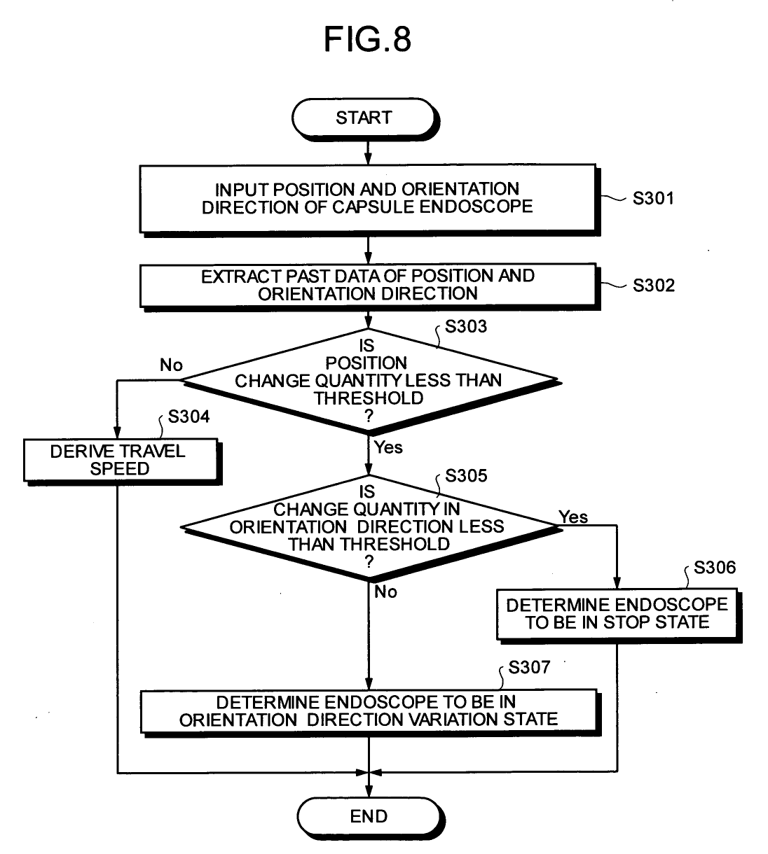

The travel state deriving operation of the capsule endoscope 2 based on the derived position and the orientation direction is explained. FIG. 8 is a flowchart of the travel state deriving operation conducted by the travel state information generator 32. Hereafter, the travel state deriving operation is explained with reference to FIG. 8 suitably.

First, the travel state information generator 32 receives information concerning the position of the capsule endoscope 2 calculated by the position calculator 39 and information concerning the orientation direction of the capsule endoscope 2 derived by the orientation direction detector 41 (step S301). The travel state information generator 32 extracts past data concerning the position and the orientation direction of the capsule endoscope 2 previously stored, to compare the past data with the received information concerning the position and the orientation direction (step S302).

Thereafter, the travel state information generator 32 calculates a position change quantity (=travel distance) of the capsule endoscope 2, and determines whether the calculated position change quantity is less than a predetermined threshold (step S303). If the position change quantity is determined to be less than the threshold, the processing proceeds to step S305. If the position change quantity is determined to be at least the threshold, the processing proceeds to step S304.

If the position change quantity is determined to be at least the threshold, derivation of the travel speed of the capsule endoscope 2 is further conducted (step S304). The system according to the present embodiment has a configuration in which the function executing unit 15 in the capsule endoscope 2 is provided with the image pickup function. When deriving the image pickup interval, therefore, the travel speed of the capsule endoscope 2 is referred to. When the derivation of the travel speed is finished, then the derivation result is fixed as the travel state information, output to the multiplexing circuit 33, and transmitted to the capsule endoscope 2 as a radio signal, and all processing is finished.

On the other hand, if the position change quantity is determined to be less than a threshold, the travel state information generator 32 derives a change quantity in the orientation direction of the capsule endoscope 2, and determines whether the derived change quantity is less than a threshold (step S305). If the change quantity in the orientation direction is determined to be less than the threshold, then the travel state information generator 32 determines the capsule endoscope 2 to be in the stop state (step S306), generates travel state information to that effect, and outputs to the multiplexing circuit 33.

If the change quantity in the orientation direction is determined to be at least the threshold at the step S305, the travel state information generator 32 determines that the capsule endoscope 2 is in an orientation direction variation state (step S307), generates travel state information to that effect, and outputs to the multiplexing circuit 33. As heretofore explained, the travel state information generator 32 determines the travel speed of the capsule endoscope 2, whether the capsule endoscope 2 is not traveling but its orientation direction is varying, or whether the capsule endoscope 2 has completely stopped its travel, based on the result of the position detection and the orientation direction detection. The travel state information generator 32 outputs a result of the determination as travel state information. Advantages of the system according to the present embodiment will be explained. First, the system according to the present embodiment calculates the position of the capsule endoscope 2 based on the constant magnetic field output by the permanent magnet 25 in the capsule endoscope 2. Unlike electromagnetic waves, the constant magnetic field has a characteristic in which the intensity attenuates nearly uniquely regardless of variations in physical parameters such as the dielectric constant and permeability in the propagation region. Therefore, the system according to the present embodiment has a feature that the relation represented by the equation (1) holds satisfactorily. Even in position detection in a space where objects such as internal organs that differ from each other in physical parameters are present as within a human body, the system according to the present embodiment has an advantage that the position can be detected with high precision as compared with the position detection using electromagnetic waves or the like.

The system according to the present embodiment has a configuration in which the orientation direction of the capsule endoscope 2 is detected based on the constant magnetic field output from the permanent magnet 25. In the same way as the position detection, the constant magnetic field output from the permanent magnet 25 is not only hardly susceptible to the influence of components in the subject 1, but also has a characteristic that the magnetic field direction in a predetermined position is determined depending nearly uniquely on the orientation direction of the capsule endoscope 2 and the relative position with respect to the capsule endoscope 2. The direction of the lines of magnetic force output from the permanent magnet 25 has a characteristic that it is nearly uniquely determined regardless of the presence of objects such as internal organs that differ from each other in physical parameters. Even while the capsule endoscope 2 is traveling in the subject 1, therefore, the orientation direction can be derived accurately by deriving the orientation direction based on the travel direction of the constant magnetic field output from the permanent magnet 25.

The system according to the present embodiment derives the travel state of the capsule endoscope 2 based on the accurately calculated position and orientation direction of the capsule endoscope 2. Owing to such a function, there is an advantage that, for example, the doctor or the like can grasp how the capsule endoscope 2 travels within the subject 1.

In the present embodiment, the drive state of the function executing unit 15 is controlled based on the derived travel state of the capsule endoscope 2. In the present embodiment, the function executing unit 15 has the illumination function and the image pickup function. Images within the subject 1 can be acquired efficiently by controlling the drive state of the function executing unit 15 based on the travel state. For example, if travel state information to the effect that the capsule endoscope 2 has stopped traveling in the subject 1 is obtained, it is possible to prevent duplicated image data of the same region from being acquired, by stopping the drive of the LED 11 and the CCD 13. When the capsule endoscope 2 travels faster than the ordinary speed as in passing through the esophagus in the subject 1, it becomes possible to acquire sufficient image data by narrowing the intervals between the image pickup operations.

In the present embodiment, the change in the orientation direction of the capsule endoscope 2 is also derived as the travel state information. For example, When the orientation direction of the capsule endoscope 2 is changing although it stays in the same region, therefore, the situation can be detected. In such a travel state, the field of vision in image pickup of the CCD 13 varies although there is no position variation of the capsule endoscope 2, and consequently more information in the subject 1 can be acquired by conducting the image pickup operation. In the system according to the present embodiment, it becomes possible to implement a configuration in which the image pickup operation is conducted, for example, when it has determined that the capsule endoscope 2 stops in the subject 1 and the change quantity in orientation direction is at least a predetermined threshold. As a result, the system according to the present embodiment has an advantage that more information can be acquired with regard to the internal images of the subject 1.

In the system according to the present embodiment, the magnetic detectors 6a to 6h are used with regard to the position detection and the orientation direction detection of the capsule endoscope 2. Therefore, there is an advantage that the system can be implemented with a simple configuration. In other words, the system according to the present embodiment detects the position and the orientation direction of the capsule endoscope 2 by using the common detector without using respective different mechanisms. Therefore, it becomes possible to form the detecting mechanism simply, resulting in an advantage of a reduced manufacture cost.

A variant of the system according to the present embodiment will be explained. The system according to the present variant relates to a test capsule used when conducting preliminary inspection of the inside of the subject to check the presence of an isthmus or the like that would hamper a smooth travel of the capsule endoscope. In other words, the system according to the present variant is provided to check how the test capsule travels in the subject.

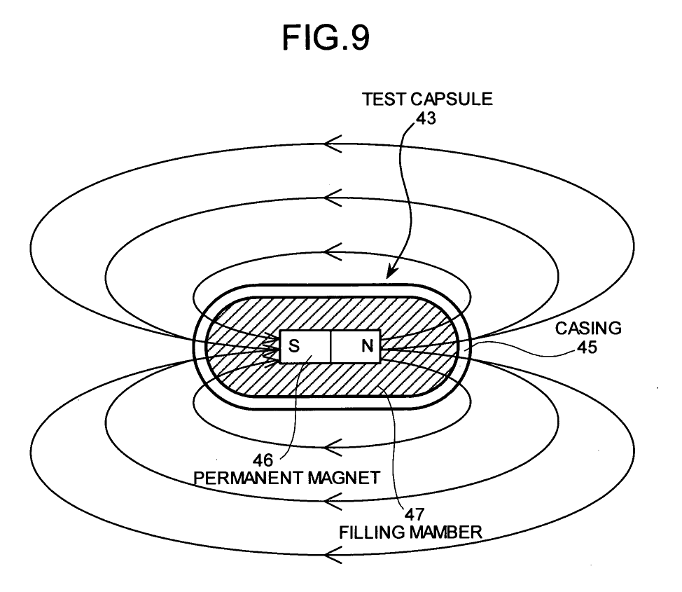

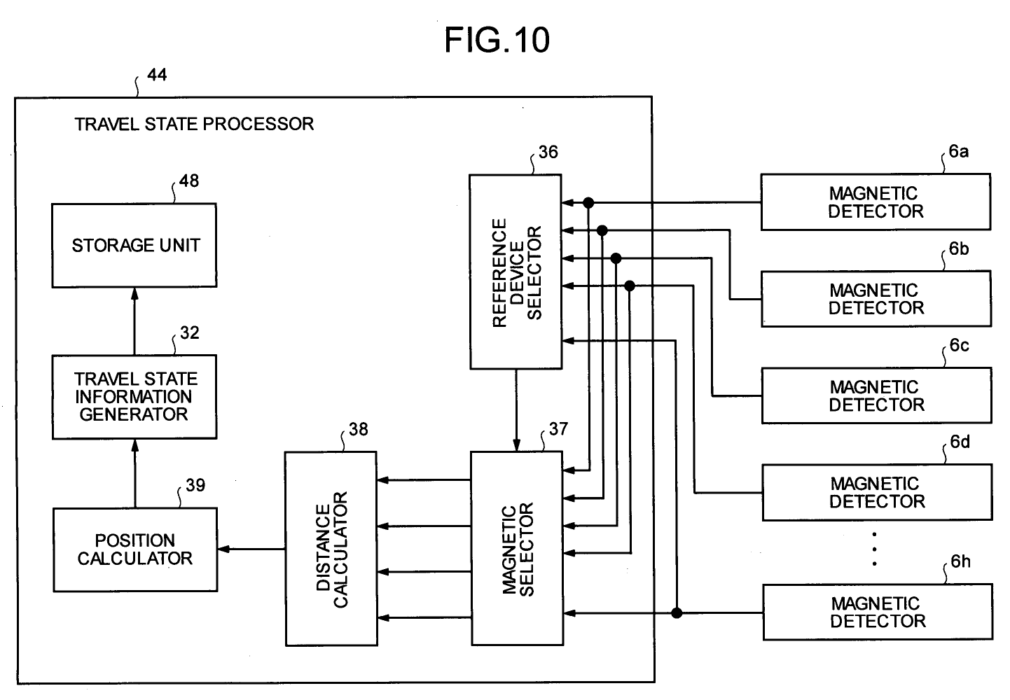

FIG. 9 is a schematic diagram of a general configuration of a test capsule 43 as a component of the system according to the present variant. FIG. 10 is a schematic diagram of a general configuration of a travel state detector included in the system according to the present variant.

As shown in FIG. 9, the test capsule 43 includes a casing 45 having a capsule shape similar to the casing of the capsule endoscope, a permanent magnet 46 disposed within the casing 45, and a filling member 47 functioning as a material that fills a clearance between the casing 45 and the permanent magnet 46.

The casing 45 is formed of a soft material that can be deformed. The casing 45 has a characteristic of being resolved when it stays in the subject 1 for a certain period of time. The configuration in which the casing 45 is resolved in the subject 1 brings about an advantage that it is not necessary to conduct an abdominal operation on the subject-1 even if the test capsule 43 introduced into the subject 1 should not be excreted to the outside of the subject 1.

The filling member 47 functions to fill the clearance between the casing 45 and the permanent magnet 46. The material forming the filling member 47 needs to be a material that does not exert a negative influence upon the subject 1. It is desirable to use, for example, a physiological saline solution or barium sulphate as the material. In particular, when the filling member 47 is formed of barium sulphate, there is an advantage that the position of the test capsule 43 can be detected by X-ray inspection using the filling member 47 as a contrast medium.

A travel state processor 44 will be explained. As shown in FIG. 10, the travel state processor 44 includes a reference device selector 36 that selects a reference device from among the magnetic detectors 6a to 6h, a magnetic selector 37 that selects selected devices based on the selected reference device and outputs a magnetic field intensities obtained by the reference device and the selected devices, a distance calculator 38 that calculates the distance from the test capsule 43 based on the magnetic field intensities output from the magnetic selector 37, a position calculator 39 that calculates the position of the test capsule 43 based on the calculated distance, and a travel state information generator 32 that generates travel state information based on the calculated position. The travel state processor 44 further includes a storage unit 48 that stores the travel state information generated by the travel state information generator 32. Owing to a configuration that outputs travel state information to the display 4 via the storage unit 48 and the portable recording medium 5, it becomes possible for the doctor or the like to grasp the travel state of the test capsule 43. In the present variant, the derived travel state information is only the positional change. As a matter of course, however, the change in the orientation direction may also be derived in the same way as the embodiment.

Heretofore, the present invention is explained with reference to the embodiment and the variant. However, the present invention is not limited to the embodiment and the variant. Various embodiments, variants and applications can occur to those skilled in the art. For example, the system according to the present embodiment may have a configuration that derives the orientation direction of the capsule endoscope 2 in the same way as the embodiment.

In the configurations of the embodiment and the variant, a plurality of magnetic detectors 6 are disposed on the outer surface of the subject 1 so as to respectively form vertexes of a cube. However, it is not necessary to limit the configuration to such an arrangement. In other words, as for the magnetic detectors 6 or the like, it is sufficient as long as the relative positions with respect to the subject 1 are previously grasped. Even if the magnetic detectors 6 are not disposed in a cubic form, the position and the orientation direction can be detected by using the relative positions. As for the number of the magnetic detectors 6 as well, it is not necessary that the number is restricted to eight. As the simplest configuration, a system using a single magnetic field detector 6 can be constructed. The capsule endoscope 2, as a device to be introduced into the subject, does not travel arbitrarily in the subject 1, but travels on a route predetermined to some degree depending on the arrangement of internal organs such as the esophagus, stomach, the small intestine, and the colon. Therefore, it is possible to previously grasp the travel route of the device. The position of the device may be detected by using the previously grasped route information and the intensity of the constant magnetic field received by the single magnetic detector.

In the embodiment and the variant, the reference device and the selected devices are selected by using the reference device selector 36 and the magnetic selector 37, and position detection is conducted based on the magnetic field intensities detected by them. However, such a configuration is not indispensable for the present invention. For example, it is possible to calculate the distances from the capsule endoscope 2 to each of the magnetic detectors 6a to 6h based on the detected intensities, form eight equations similar to Equations (2) to (5), and calculate the position of the capsule endoscope 2. In such a configuration, for example, computation using the least square method is possible and this results in an advantage that the derivation error in the position of the capsule endoscope 2 can be further reduced.

In the same way, for example, in the embodiment, the orientation direction of the capsule endoscope 2 may be derived by using a plurality of magnetic detectors 6. In other words, it is also desirable to adopt a configuration in which the derivation of the orientation direction is conducted in the manner as described above at a plurality of magnetic detectors 6 to find an average of the obtained orientation directions, for example, thereby allowing a more accurate derivation of orientation direction. The same holds for the position detection of the device to be introduced into the subject as well. It is possible to adopt a configuration in which position detection is conducted a plurality of times by using different combinations of the magnetic detectors and respectively obtained positions are averaged.

Further, in the embodiment, the function executing unit 15 including the CCD 13 serving as the image capturing unit and the LED 11 serving as the illuminating unit is explained. However, the function executing unit may have a configuration as to acquire information concerning pH and the temperature in the subject 1. The device to be introduced into the subject may have a oscillator to acquire an ultrasonic wave image in the subject 1. Still further, the device may have a configuration to acquire a plurality of pieces of information from the intra-subject information.

The radio signal transmitted from the transmitting antennas 10-1 to 10-m need not be the travel state information signal superposed on the power supplying signal, but a configuration that outputs only the travel state information signal may be adopted. The travel state information signal may be superposed on a signal other than the power supplying signal. The travel state detector 3 may have a configuration that conducts only reception of the radio signal output from the capsule endoscope 2. It is also possible to provide a storage unit in the capsule endoscope, and retrieve information from the storage unit after the capsule endoscope is excreted to the outside of the subject 1.

As is clear from the foregoing, the system according to the present invention is useful in connection with the swallowable capsule endoscope employed for the medical treatment, and particularly suitable for a device to be introduced into a subject, such as a patient, for the position detection.

Although the invention has been described with respect to a specific embodiment for a complete and clear disclosure, the appended claims are not to be thus limited but a re to be construed as embodying all modifications and alternative constructions that may occur to one skilled in the art which fairly fall within the basic teaching herein set forth.

1. A system comprising:
a device that is swallowed, passes through a subject, and includes a magnetic field generator generating a constant magnetic field; and
a travel state detector that includes
a magnetic detector, disposed in a fixed relative position to the subject, detecting an intensity of a constant magnetic field output from the magnetic field generator,
a position processor calculating a position of the device in the subject based on the intensity of the magnetic field detected by the magnetic detector, and
a travel state processor determining a travel state of the device in the subject based on the position calculated by the position processor.

2. The system according to claim 1, wherein the travel state detector includes a plurality of magnetic detectors, and
the position processor calculates a distance between the device and each of the magnetic detectors based on intensities of magnetic field detected by the magnetic detectors, and calculates the position of the device in the subject based on the calculated distances.

3. The system according to claim 1 wherein the magnetic field generator is disposed in a position where a direction of the constant magnetic field is fixed, and
the travel state detector further includes a magnetic filed direction detector detecting a direction of the constant magnetic field generated from the constant magnetic field generator, and an orientation direction detector detecting an orientation direction of the device in the subject based on the direction detected by the magnetic field direction detector.

4. The system according to claim 3 wherein the travel state detector further includes an orientation direction database that stores, in advance, a distance from the magnetic field generator, a relation between a direction of the constant magnetic field, and an orientation direction of the device in the subject, and
the orientation direction detector detects the orientation direction of the device in the subject using the orientation direction database.

5. The system according to claim 1 wherein the device further includes a function executing unit that obtains information of an inside of the subject, and a radio transmitting unit that transmits the information of the inside of the subject with radio communication, and
the travel state detector further includes a receiving unit that receives a radio signal transmitted from the radio transmitting unit.

6. The system according to claim 5 wherein the travel state detector generates travel state information to control a drive state of the function executing unit provided in the device based on the travel state of the device in the subject determined by the travel state processor.

7. The system according to claim 5 wherein the function executing unit includes an illuminating unit that illuminates an inside of the subject, and an image capturing unit that captures an image of a region illuminated by the illuminating unit.

8. The system according to claim 1, wherein the device is a capsule endoscope.
